(19) United States
(12) Patent Application Publication
(10) Pub. No.: US 2025/0339032 A1
(43) Pub. Date: Nov. 6, 2025

(54) MICROSCOPE OBJECTIVE LENS, ATTACHMENT FOR MICROSCOPE OBJECTIVE LENS, AND MICROSCOPE

(71) Applicant: NATIONAL INSTITUTES FOR QUANTUM SCIENCE AND TECHNOLOGY, Chiba (JP)

(72) Inventors: Ryuji IGARASHI, Chiba (JP); Kiichi KAMINAGA, Chiba (JP)

(21) Appl. No.: 18/846,572

(22) PCT Filed: Mar. 17, 2023
(86) PCT No.: PCT/JP2023/010763

§ 371 Date: May 1, 2025

(30) Foreign Application Priority Data
Mar. 17, 2022 (JP) 2022-043239

(51) Int. Cl. A61B 5/00; G02B 21/00; G02B 21/02; G02B 21/16; H01Q 1/22

(52) U.S. Cl. CPC A61B 5/0071; G02B 21/0012; G02B 21/02; G02B 21/16; H01Q 1/22

(57) ABSTRACT

An object of the present invention is to provide a microscope objective lens, microscope objective lens attachment, and microscope which enable simple, quick, accurate, highly reproducible positioning of an electromagnetic wave applying antenna in applying an electromagnetic wave under a microscope. To attain the object, provided are a microscope objective lens including an antenna configured to apply an electromagnetic wave to an observation target and provided on a front side of a lens surface thereof, a microscope objective lens attachment including an antenna, and a microscope using the microscope objective lens or a microscope objective lens with the microscope objective lens attachment attached thereto. According to the feature, the electromagnetic wave applying antenna is provided directly or indirectly to a microscope objective lens side, enabling simple, quick, accurate, highly reproducible positioning of the electromagnetic wave applying antenna according to position adjustment of the microscope objective lens and the observation target.

### TECHNICAL FIELD

The present invention relates to a microscope objective lens, a microscope objective lens attachment, and a microscope. In particular, the present invention relates to a microscope objective lens, a microscope objective lens attachment, and a microscope each of which is used for microscope observation/measurement involving application of an electromagnetic wave.

### BACKGROUND ART

Advances in microscope observation technology enable fluorescent molecule observation methods using fluorescence microscopes to carry out single-molecule measurement and real-time observation/measurement. The fluorescent molecule observation methods have been considered to be used to acquire information on the dynamics (including structure analysis) of an observation target (mainly protein) in a living body (in a cell), i.e., so-called in-vivo measurement.

Meanwhile, it is difficult to measure fluctuation and change of a structure of an observation target with a spatial resolution of a typical fluorescence microscope. Further, many phosphors, which are fluorescent molecular probes that label an observation target for observation/measurement using the fluorescent molecule observation method, are toxic to living bodies and are not suitable for non-invasive measurement.

Here, as a method that allows for non-invasive measurement in the protein structure analysis, a molecular structure analysis method using nuclear magnetic resonance (hereinafter, referred to as “NMR”) is also known.

Measurement using the NMR exhibits high spatial resolution on one hand, but on the other hand, has low sensitivity and time resolution, resulting in difficulty in real-time observation.

Meanwhile, as a method that allows for high-sensitivity detection of magnetic resonance in an observation target, measurement using optically-detected magnetic resonance (hereinafter, also referred to as “ODMR”) is known. The measurement using the ODMR is to detect magnetic resonance with high sensitivity by irradiating an observation target with excitation light and a high-frequency magnetic field simultaneously and detecting a change in an amount of fluorescence emission. Measurement using the ODMR in which the fluorescent molecule observation method using a fluorescence microscope and measurement using NMR are combined has been considered to be used to grasp dynamics of an observation target in a living body (in-vivo measurement).

For example, Non-Patent Literature 1 describes carrying out the ODMR measurement on living bodies (nematodes and mice) with use of nanodiamond particles as fluorescent molecular probes.

### CITATION LIST

### Non-Patent Literature

Non-Patent Literature 1: R. Igarashi et al., “Real-time background-free selective imaging of fluorescent nanodiamonds in vivo”, Nano letters, vol. 12, pp. 5726-5732, 2012.

### SUMMARY OF INVENTION

### Technical Problem

In the measurement using the ODMR, an antenna (radiator) for applying a high frequency (electromagnetic wave) to an observation target is required. As an antenna for applying a high frequency (electromagnetic wave) (hereinafter referred to as “electromagnetic wave applying antenna” or simply “antenna”), Non-Patent Literature 1 discloses one using a high-frequency coil. In order to accurately apply a high frequency to an observation target, it is unfortunately necessary to carry out position adjustment in each measurement so that the high-frequency coil and the observation target are disposed at optimum positions with sufficient reproducibility. The fact that this position adjustment needs to be carried out with high accuracy raises, for example, a problem of complicating the structure itself of a mechanism for adjusting the positions and a problem of being incapable of securing a space for the mechanism configured to carry out the position adjustment due to a structure of a microscope.

Other examples of the electromagnetic wave applying antenna include one formed by drawing through metal evaporation on a substrate on which an observation target is to be disposed. In this case, however, it is necessary to draw an electromagnetic wave applying antenna on a substrate for each observation target, and therefore, this is disadvantageously not suitable for handling a large number of observation targets. Further, there is a problem in that since a positional relationship between the observation target and the electromagnetic wave applying antenna is fixed, an electromagnetic wave can be applied only in a range drawn as an antenna.

Therefore, a technology is in demand which enables simple, quick, accurate, highly reproducible position adjustment in adjusting a position of an electromagnetic wave applying antenna configured to apply an electromagnetic wave to an observation target, in a case where an electromagnetic wave needs to be applied under a microscope.

Thus, it is an object of the present invention to provide a microscope objective lens, a microscope objective lens attachment, and a microscope each of which enables simple, quick, accurate, highly reproducible positioning of an electromagnetic wave applying antenna in applying an electromagnetic wave under a microscope.

### Solution to Problem

As a result of diligent study on the above object, the inventors of the present invention have found that providing an electromagnetic wave applying antenna directly or indirectly to a microscope objective lens side enables simple, quick, accurate, highly reproducible positioning of the electromagnetic wave applying antenna in accordance with position adjustment of the microscope objective lens and the observation target, and the inventors have completed the present invention.

That is, the present invention encompasses the following microscope objective lens, microscope objective lens attachment, and microscope. Note that hereinafter, the microscope objective lens attachment is also referred to simply as “attachment”.

A microscope objective lens of the present invention for attaining the foregoing object has a feature of including an antenna configured to apply an electromagnetic wave to an observation target, the antenna being provided on a front side of a lens surface of the microscope objective lens.

This feature enables a relative position between the lens surface and the electromagnetic wave applying antenna in the microscope objective lens to be constant and makes it possible to position the electromagnetic wave applying antenna with the accuracy with which the microscope objective lens is positioned. This enables simple, quick, accurate, highly reproducible positioning of the electromagnetic wave applying antenna in applying an electromagnetic wave under a microscope. Further, this enables an observation target and the electromagnetic wave applying antenna to be positioned independently of each other, thereby making it possible to apply an electromagnetic wave to a desired part of the observation target.

An embodiment of the microscope objective lens of the present invention is configured such that the antenna includes a conductive member and a fixing section that fixes the conductive member on a front side of the lens surface.

According to this feature, the conductive member is used as an antenna configured to apply (radiate) an electromagnetic wave to an observation target, so that it is possible to adjust an intensity of an electromagnetic wave to be applied and directivity of an antenna depending on selection of a size (area, thickness, etc.) of the conductive member and selection of an antenna shape formed by the conductive member. Further, a position and height of the conductive member can be fixed by providing a fixing section that fixes the conductive member, thereby enabling a distance between the electromagnetic wave applying antenna and the lens surface of the microscope objective lens, and a distance between the electromagnetic wave applying antenna and the focal plane of the observation target to be stably kept constant. This makes it possible to effectively apply an electromagnetic wave to an observation target.

An embodiment of the microscope objective lens of the present invention is configured such that the antenna includes a pattern drawn through metal evaporation on a light transmissive substrate.

This feature makes it possible to easily form an antenna having a desired shape. Further, a failure such as disconnection hardly occurs, thereby making it possible to configure an antenna having high durability against repeated use.

A microscope objective lens attachment of the present invention for attaining the foregoing object is a microscope objective lens attachment attached to a microscope objective lens, the microscope objective lens attachment including: a tubular body; a first opening part that enables the microscope objective lens to be inserted therethrough into the tubular body; and a second opening part that exposes, to an outside, a lens surface of the microscope objective lens inserted into the tubular body, the microscope objective lens attachment being provided with an antenna that is formed on a second opening part side and that is configured to apply an electromagnetic wave to an observation target.

This feature enables a relative position of the microscope objective lens with the attachment attached thereto and the electromagnetic wave applying antenna to be constant and makes it possible to position the electromagnetic wave applying antenna with the accuracy with which the microscope objective lens is positioned. This enables simple, quick, accurate, highly reproducible positioning of the electromagnetic wave applying antenna in applying an electromagnetic wave under a microscope. Further, this enables an observation target and the electromagnetic wave applying antenna to be positioned independently of each other, thereby making it possible to apply an electromagnetic wave to a desired part of the observation target.

Furthermore, according to this feature, the attachment is configured to be attachable to a microscope objective lens, thereby enabling an existing microscope objective lens to be easily switched between a mode suitable for microscope observation involving application of an electromagnetic wave and a mode suitable for ordinary microscope observation.

An embodiment of the microscope objective lens attachment of the present invention is configured such that the antenna includes a conductive member and a support section supporting the conductive member.

According to this feature, the conductive member is used as an antenna configured to apply (radiate) an electromagnetic wave to an observation target, so that it is possible to easily adjust an intensity of an electromagnetic wave to be applied and directivity of an antenna with a simple process, such as change of a size (area, thickness, etc.) of the conductive member and change of an antenna shape formed by the conductive member. Further, the support section that supports the conductive member is provided, so that it is possible to fix a position and height of the conductive member on the attachment, thereby enabling a distance between the electromagnetic wave applying antenna and the lens surface of the microscope objective lens, and a distance between the electromagnetic wave applying antenna and the focal plane of the observation target to be stably kept constant. This makes it possible to effectively apply an electromagnetic wave to an observation target.

An embodiment of the microscope objective lens attachment of the present invention is configured such that the conductive member extends to a side surface of the tubular body.

In order to cause a conductive member to act as an electromagnetic wave applying antenna, it is necessary to transmit an electromagnetic wave to the conductive member, more specifically, it is necessary to cause the conductive member and an oscillator configured to transmit an electromagnetic wave to the conductive member to be connected to (brought into contact with) each other. However, when a working distance of the objective lens is short, it is not easy to cause an antenna (conductive member) provided on an upper surface of the objective lens and an oscillator to be connected to (brought into contact with) each other on the same plane.

Meanwhile, according to this feature, the conductive member extends to a side surface of the body of the attachment, so that it is possible to cause the conductive member and the oscillator to be connected to (brought into contact with) each other at a part on a side surface side of the body of the attachment, that is, on a side surface side of the housing of the microscope objective lens. This makes it easy to, even in a case where a working distance of an objective lens is short, secure a space that allows the conductive member and the oscillator to be connected to (brought into contact with) each other.

An embodiment of the microscope objective lens attachment of the present invention is configured such that the antenna includes a pattern drawn through metal evaporation on a light transmissive substrate.

This feature makes it possible to easily form an antenna having a desired shape. Further, a failure such as disconnection hardly occurs, thereby making it possible to configure an antenna having high durability against repeated use.

An embodiment of the microscope objective lens attachment of the present invention is configured such that the tubular body includes a slit in a circumferential direction.

This feature has an advantage that operation of the correction collar disposed on the housing of the microscope objective lens is not inhibited.

A microscope of the present invention for attaining the foregoing object includes the above-described microscope objective lens provided with an antenna on a front side of a lens surface thereof and an oscillator configured to transmit an electromagnetic wave to the antenna of the microscope objective lens.

This feature enables observation/measurement using a microscope objective lens that allows for simple, quick, accurate, highly reproducible positioning of the electromagnetic wave applying antenna in carrying out observation/measurement involving application of an electromagnetic wave under a microscope. Further, this enables an observation target and the electromagnetic wave applying antenna to be positioned independently of each other, thereby making it possible to apply an electromagnetic wave to a desired part of the observation target.

Another aspect of the microscope of the present invention for attaining the foregoing object includes a microscope objective lens, the above-described microscope objective lens attachment, and an oscillator configured to transmit an electromagnetic wave to the antenna of the microscope objective lens attachment.

According to this feature, attaching the microscope objective lens attachment enables observation/measurement using the microscope objective lens that allows for simple, quick, accurate, highly reproducible positioning of an electromagnetic wave applying antenna in carrying out observation/measurement involving application of an electromagnetic wave under a microscope. Further, this enables an observation target and the electromagnetic wave applying antenna to be positioned independently of each other, thereby making it possible to apply an electromagnetic wave to a desired part of the observation target.

Further, according to the feature, the attachment attachable to a microscope objective lens is attached to and detached from the microscope objective lens, so that it is possible to use a microscope as a microscope that can be easily switched between a mode suitable for microscope observation involving application of an electromagnetic wave and a mode suitable for ordinary microscope observation.

### Advantageous Effects of Invention

According to the present invention, it is possible to provide a microscope objective lens, a microscope objective lens attachment, and a microscope each of which enables simple, quick, accurate, highly reproducible positioning of an electromagnetic wave applying antenna in applying an electromagnetic wave under a microscope.

### BRIEF DESCRIPTION OF DRAWINGS

FIG. 1A is an explanatory side view schematically illustrating a structure of a microscope objective lens in accordance with Embodiment 1 of the present invention.

FIG. 1B is an explanatory plan view when seen from above of the microscope objective lens in accordance with Embodiment 1 of the present invention.

FIG. 1C is an explanatory perspective view of the microscope objective lens in accordance with Embodiment 1 of the present invention. FIG. 2A is an explanatory side view schematically illustrating another aspect of an antenna in the microscope objective lens in accordance with Embodiment 1 of the present invention.

FIG. 2B is an explanatory plan view when seen from above schematically illustrating another aspect of an antenna in the microscope objective lens in accordance with Embodiment 1 of the present invention.

FIG. 3 is an explanatory view schematically illustrating a structure of a microscope in accordance with Embodiment 1 of the present invention.

FIG. 4 is an explanatory view schematically illustrating another aspect of the microscope in accordance with Embodiment 1 of the present invention.

FIG. 5A is an explanatory view (side view) schematically illustrating structures of a microscope objective lens and a microscope objective lens attachment in accordance with Embodiment 2 of the present invention.

FIG. 5B is an explanatory view (a plan view when seen from above) schematically illustrating a structure of the microscope objective lens attachment in accordance with Embodiment 2 of the present invention.

FIG. 5C is an explanatory view (perspective view) schematically illustrating a structure of the microscope objective lens attachment in accordance with Embodiment 2 of the present invention.

FIG. 6 is an explanatory view (side view) schematically illustrating another aspect of the microscope objective lens attachment in accordance with Embodiment 2 of the present invention.

FIG. 7 is an explanatory view schematically illustrating a structure of a microscope in accordance with Embodiment 2 of the present invention.

FIG. 8 is a typical image of ODMR measurement carried out with use of the microscope in accordance with Embodiment 2 of the present invention.

FIG. 9 is a graph indicating a signal intensity with respect to a swept frequency in ODMR measurement carried out with use of the microscope in accordance with Embodiment 2 of the present invention.

### DESCRIPTION OF EMBODIMENTS

A microscope objective lens, a microscope objective lens attachment, and a microscope of the present invention are used for microscope observation/measurement involving operation of applying an electromagnetic wave to an observation target. Although the type of microscope observation to which the present invention is applied is not particularly limited, but it is suitably used in particular for microscope observation/measurement using ODMR.

Here, an electromagnetic wave applied by an antenna of the present invention is selected/set as appropriate in accordance with of the type the microscope observation/measurement. In this case, it is possible that an electromagnetic wave applied from an antenna to an observation target is set, and it is also possible that an electromagnetic wave transmitted to an antenna is set. For example, in the microscope observation/measurement using ODMR, one possible setting is such that a microwave (a frequency of 300 MHz to 30 GHz) or a radio wave (a frequency of 30 to 300 MHz) can be applied to an antenna.

The observation target, which is a target of observation/measurement through microscope observation using each of a microscope objective lens, a microscope objective lens attachment, and an a microscope of the present invention is not particularly limited. Examples of such an observation target include, as ones known to be suitable as targets of observation/measurement (structure analysis) involving application of an electromagnetic wave, minerals, such as iron ore, and protein. In particular, for the observation target of the present invention, it is preferable to use a fluorescent probe and regard protein in a living body as an observation target.

The following will describe an embodiment of a microscope objective lens, an embodiment of a microscope objective lens attachment, and an embodiment of a microscope of the present invention in detail with reference to the drawings.

Note that the microscope objective lens, the microscope objective lens attachment, and the microscope which are described as embodiments are merely taken as examples for describing the present invention, and each of these should not be construed as a limitation.

### Embodiment 1

### Microscope Objective Lens

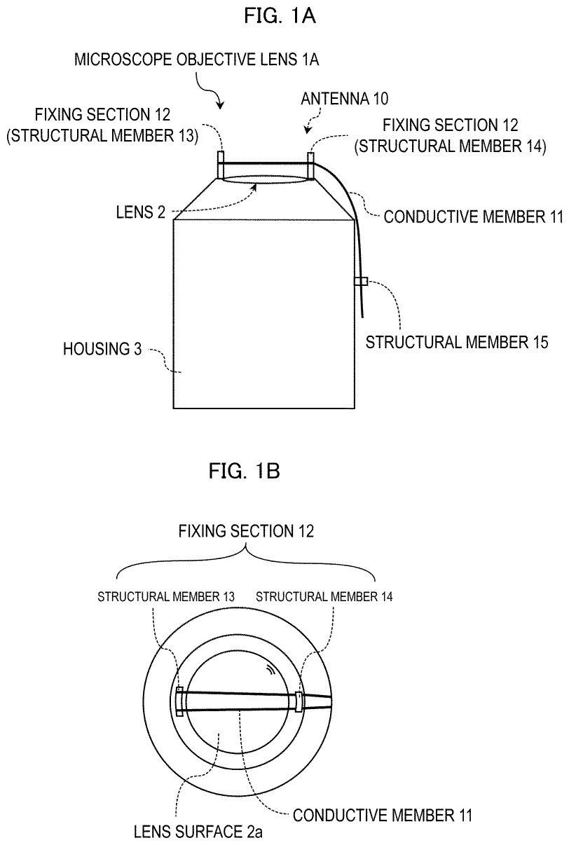

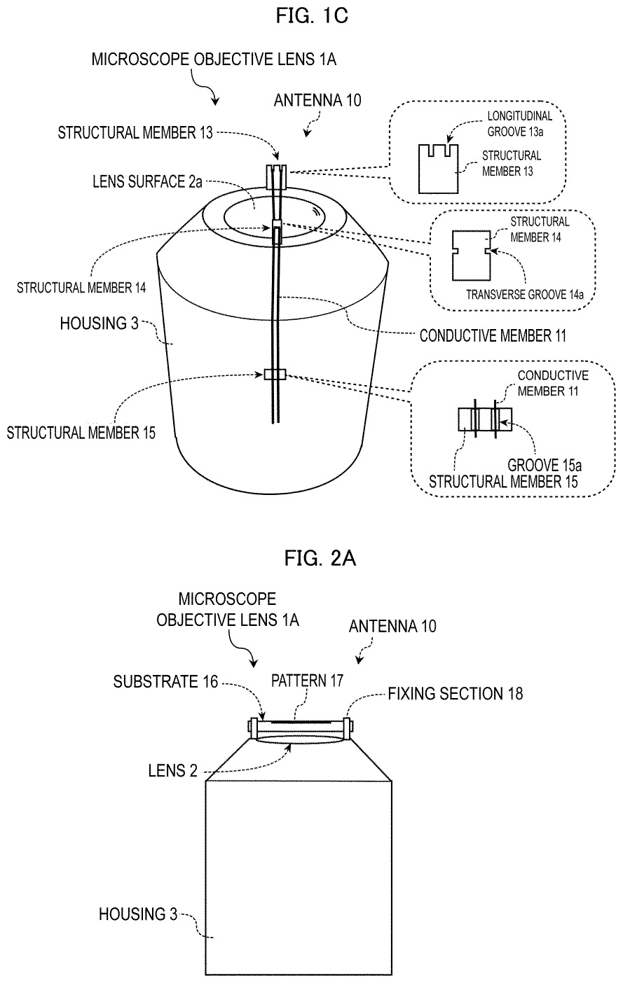

FIGS. 1A, 1B and 1C are explanatory views schematically illustrating a structure of a microscope objective lens in accordance with Embodiment 1 of the present invention. FIG. 1A is a side view, FIG. 1B is a plan view when seen from above, and FIG. 1C is a perspective view.

As illustrated in FIG. 1A, a microscope objective lens 1A in accordance with the present embodiment includes a lens 2, a housing 3, and an antenna 10 configured to apply an electromagnetic wave to an observation target S. Here, the lens 2 refers to the lens disposed closest to the observation target S.

Known configurations as a microscope objective lens can be used for the lens 2 and the housing 3 in the microscope objective lens 1A in accordance with the present embodiment. The microscope objective lens 1A, for example, only needs to include at least the lens 2 facing the observation target S, and may be made up from a single lens or may include a plurality of lenses in the housing 3. Further, a known configuration as a microscope objective lens may be included in addition to the lens 2 and the housing 3. For example, the microscope objective lens 1A may be provided with a correction collar (unillustrated), which is a mechanism configured to make correction by moving the lens inside the housing 3 in a direction of an optical axis.

The antenna 10 in the microscope objective lens 1A in accordance with the present embodiment is configured to apply an electromagnetic wave to the observation target S.

As illustrated in FIG. 1, the antenna 10 in accordance with the present embodiment is provided on a front side of the lens 2 (lens surface 2a facing the observation target S), which corresponds to an upper side of the lens 2 (lens surface 2a) in FIG. 1A.

The antenna 10 only needs to be one that can receive an electromagnetic wave from an oscillator 200 provided in a microscope 100A described later and can apply the electromagnetic wave to the observation target S.

One example of the antenna 10 in accordance with the present embodiment is one including a conductive member 11 and a fixing section 12 that fixes the conductive member 11 on a front side of the lens surface 2a as illustrated in FIG. 1A.

The conductive member 11 only needs to be made of material capable of transmitting and radiating an electromagnetic wave. Examples of such a material include a plate-shaped member made of known conductive material, as well as a metal line (wire) made of, for example, copper, silver, or gold. Further, one obtained by coating a metal line with a coating material may be used in order to increase the strength. Note that the conductive member 11 in accordance with the present embodiment will be described taking one mainly made from a metal line (wire) as an example, as illustrated in FIG. 1A, but this should not be construed as a limitation.

In this case, a size (area, length, thickness, etc.) of the conductive member 11 can be selected as appropriate from among ones that enable transmission and radiation of a desired electromagnetic wave, and is not particularly limited. For example, in a case where a metal line is used as the conductive member 11, the antenna 10 and the observation target S are spaced from each other by a radius of the metal wire. Therefore, in order to effectively apply an electromagnetic wave to the observation target S, it is preferable to use a thin (small diameter) metal line. More specifically, the diameter of the metal line used as the conductive member 11 is preferably not more than 1 mm, more preferably not more than 500 μm, and still more preferably not more than 50 μm.

Further, a shape of the antenna 10 formed by the conductive member 11 is not particularly limited, but it is preferable to employ a shape such that directivity as the antenna 10 is considered and such that the conductive member 11 does not interfere in an observation field of view in microscope observation. For example, as illustrated in FIG. 1B, one possible shape is a ring coil (loop coil) formed by bridging the conductive member 11 (metal line) on a front side of the lens surface 2a with use of the fixing section 12 described later. Thus, an electromagnetic wave is applied (radiated) to an inside of the ring coil, making it possible to effectively apply an electromagnetic wave to the observation target S in an observation field of view.

The fixing section 12 is configured to fix the conductive member 11 on a front side of the lens surface 2a.

The fixing section 12 in accordance with the present embodiment only needs to be one that can fix the conductive member 11 on a front side of the lens surface 2a, and a specific structure thereof is not particularly limited. For the fixing section 12, a configuration is possible in which a structural member 13 and a structural member 14 that enable the conductive member 11 (metal line) to be bridged therebetween are provided on the housing 3 in the vicinity of an outer periphery of the lens 2 so as to face each other, for example, as illustrated in FIG. 1B. This makes it possible to easily form a shape of the antenna 10 which can effectively apply an electromagnetic wave to the observation target S without the conductive member 11 interfering in an observation field of view.

In this case, as illustrated in FIG. 1C, the structural member 13 and the structural member 14 are preferably provided with longitudinal grooves 13a and transverse grooves 14a, respectively. This makes it easy to bridge the conductive member 11 (metal line) without slack, thus making it possible to fix a position of the conductive member 11. The structural member 13 and the structural member 14 each may include any structure that makes it easy to bridge and fix the conductive member 11, and the structure is not limited to the longitudinal grooves 13a and the transverse grooves 14a. For example, another possible structure is such that holes are provided in the structural member 13 and the structural member 14, and the conductive member 11 (metal line) is bridged through the hole of each structure, to fix the position of the conductive member 11, instead of providing the grooves in the structural member 13 and the structural member 14.

Selection and design of heights of the structural member 13 and the structural member 14 as the fixing section 12 (a height at which the conductive member 11 is bridged) enables a distance between the lens surface 2a and the antenna 10 to be constantly kept a desired height (length). At the same time, it is possible to keep a distance between the focal plane of the observation target S and the antenna 10 constant at the time of microscope observation.

Here, an excessive distance between the observation target S and the antenna 10 raises a problem in that an electromagnetic wave from the antenna 10 is not appropriately applied to the observation target S. Therefore, the heights of the structural member 13 and the structural member 14 (a height at which the conductive member 11 is bridged) are preferably set so that a distance between the observation target S and the antenna 10 is not more than 20 mm and are more preferably set so that the distance is not more than 5 mm.

In the setting of the heights of the structural member 13 and the structural member 14, there is no particular limitation on how close the distance between the observation target S and the antenna 10 is. For example, the setting may be made so that the distance between the observation target S and the antenna 10 is zero. In this case, since an electromagnetic wave generated from the antenna 10 is transferred also as heat to the observation target S, there is a risk that the observation target S is altered. Therefore, in a case where the distance between the observation target S and the antenna 10 is close, it is preferable to adjust (weaken) an intensity of the electromagnetic wave to be applied.

Meanwhile, the widths of the structural member 13 and the structural member 14 are not particularly limited. However, as a trend of the performance of an antenna made from a ring coil, it is known that when the diameter of the ring coil (corresponding to each width of the structural member 13 and the structural member 14 in accordance with the present embodiment) becomes wide to a certain extent, efficiency of applying an electromagnetic wave decreases. In contrast, when the widths of the structural member 13 and the structural member 14 in accordance with the present embodiment are made narrower, the conductive member 11 bridged therebetween comes to interfere in an observation field of view in the microscope observation. Therefore, the widths of the structural member 13 and the structural member 14 are each preferably designed to maintain a width enough to prevent the conductive member 11 bridged therebetween from interfering in the observation field of view.

In a case where the conductive member 11 is used as the antenna 10, the conductive member 11 is preferably caused to extend to a side surface of the housing 3. As described later, in order to cause the conductive member 11 to act as the antenna 10 configured to apply an electromagnetic wave, it is necessary to transmit an electromagnetic wave to the conductive member 11, more specifically, it is necessary to cause the conductive member 11 and the oscillator 200 configured to transmit an electromagnetic wave to the conductive member 11 to be connected to (brought into contact with) each other. However, in a case where a working distance as the microscope objective lens 1A is short, it is difficult to cause the conductive member 11 provided on a lens surface 2a side and the oscillator 200 to be connected to (brought into contact with) each other on the same plane.

Meanwhile, when the conductive member 11 extends to the side surface of the housing 3, it is possible to cause the conductive member 11 and the oscillator 200 to be connected to (brought into contact with) each other at a part on a side surface side of the housing 3. This makes it easy to, even in a case where a working distance as the microscope objective lens is short, secure a space that allows the conductive member 11 and the oscillator 200 to be connected to (brought into contact with) each other.

In this case, as illustrated in FIG. 1C, it is preferable to provide a structural member 15 having grooves 15a to a side surface of the housing 3 to fix the conductive member 11 extending to the side surface of the housing 3. This enables the conductive member 11 and the oscillator 200 to be stably connected to (brought into contact with) each other.

Note that the fixing section 12 is not limited to the structural members 13 to 15 illustrated in FIGS. 1A to 1C. For example, in a case where a plate-shaped member is used as the conductive member 11, it is possible to use, as the fixing section 12, a structural member including a structure (for example, a locking part, a claw part, or a clamping part) that fixes the plate-shaped member, instead of the structural members 13 to 15 each including the grooves (the longitudinal grooves 13a, the transverse grooves 14a, or the grooves 15a).

Further, the antenna 10 in accordance with the present embodiment is not limited to the one using the conductive member 11.

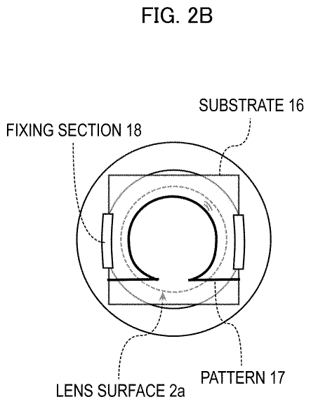

FIGS. 2A and FIG. 2B are explanatory views schematically illustrating another aspect of the antenna 10 in a microscope objective lens 1B in accordance with the present embodiment. FIG. 2A is a side view, and FIG. 2B is a plan view when seen from above.

As illustrated in FIG. 2B, another possible aspect of the antenna 10 is to use one including a pattern 17 drawn on a light transmissive substrate 16.

In this case, the pattern 17 may be made of metal and be drawn on the substrate 16 through metal evaporation, specifically, by +.

Note that the substrate 16 on which the pattern 17 is drawn only needs to be made of light transmissive material that transmits light (wavelength) used in microscope observation. Examples of such material include a glass plate, which has a high light transmission capability and is easily available and processed.

The antenna 10 is configured as the pattern 17 drawn on the substrate 16, so that it is possible to easily form an antenna having a desired shape. Further, a failure such as disconnection hardly occurs, thereby making it possible to configure an antenna having high durability against repeated use.

The substrate 16 on which the pattern 17 is drawn is disposed on a front side of the lens surface 2a (corresponding to an upper side of the lens surface 2a in FIG. 2A). The substrate 16 may be fixed to the lens surface 2a or may be provided with a fixing section 18 that fixes the substrate 16 to the housing 3 at an outer periphery of the lens 2, as illustrated in FIG. 2A.

The pattern 17 drawn on the substrate 16 has any shape that is capable of applying an electromagnetic wave to the observation target S, and there is no particular limitation on the shape. The shape is however preferably set on the basis of, for example, a frequency of the electromagnetic wave to be transmitted/applied and desired directivity as the antenna 10. Further, the pattern 17 may have any shape that does not interfere in the observation field of view in the microscope observation, and the shape of the pattern 17 may have a part overlapping the lens surface 2a. Examples of a specific shape of the pattern 17 include a shape having a ring-shaped (loop-shaped) part along a circumference of the lens surface 2a as illustrated in FIG. 2B, and a linear shape.

As described above, the microscope objective lens 1A in accordance with the present embodiment is provided directly with the antenna 10, thereby enabling a relative position of the lens surface 2a of the microscope objective lens and the antenna 10 to be constant and enabling positioning of the antenna 10 with the accuracy with which the microscope objective lens 1A is positioned. This enables simple, quick, accurate, highly reproducible positioning of the electromagnetic wave applying antenna (antenna 10) in applying an electromagnetic wave under a microscope. Further, this enables the observation target S and the electromagnetic wave applying antenna (antenna 10) to be positioned independently of each other, thereby making it possible to apply an electromagnetic wave to a desired part of the observation target S.

The microscope objective lens 1A in accordance with the present embodiment is suitably used for microscope observation/measurement involving application of an electromagnetic wave. In particular, it is suitably used as a microscope objective lens for the ODMR measurement.

### Microscope

The above-described microscope objective lens 1A can applied be to a microscope that carries out observation/measurement by applying an electromagnetic wave to the observation target S.

The following will show an example in which the microscope objective lens 1A is applied to an optical microscope (fluorescence microscope) that carries out ODMR measurement, as a microscope of the present invention to which the microscope objective lens 1A is applied.

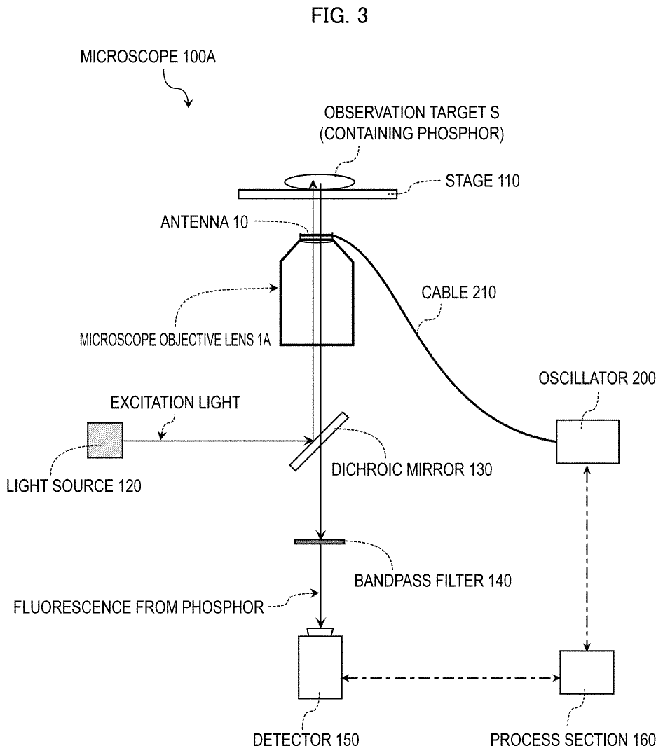

FIG. 3 is an explanatory view schematically illustrating a structure of a microscope in accordance with Embodiment 1 of the present invention.

As illustrated in FIG. 3, a microscope 100A in accordance with the present embodiment includes the microscope objective lens 1A including the antenna 10 described above, and the oscillator 200 configured to transmit an electromagnetic wave to the antenna 10. As the microscope objective lens 1A, FIG. 3 shows one that uses the conductive member 11 (metal line) illustrated in FIG. 1A as the antenna 10, but this should not be construed as a limitation. It is possible to use ones in accordance with the structures described as the microscope objective lens 1A above.

One possible microscope 100A in accordance with the present embodiment is one including a stage 110 on which the observation target S is to be placed, a light source 120, a dichroic mirror 130, a bandpass filter 140, a detector 150, and a process section 160, as a typical structure as an optical microscope (fluorescence microscope). Here, the microscope 100A illustrated in FIG. 3 is shown as one example as the microscope 100A in accordance with the present embodiment, and does not limit how the components are disposed. Further, the microscope 100A illustrated in FIG. 3 shows a structure of an inverted microscope in which the microscope objective lens 1A is disposed under the observation target S, this should not be construed as a limitation. It is also possible to use a structure of an upright microscope in which the microscope objective lens 1A is disposed above the observation target S.

Note that in FIG. 3, the arrow shown by a solid line indicates an optical axis, and the arrow shown by a dot-and-dash line indicates connection that enables input and output.

The observation target S observed with use of the microscope 100A in accordance with the present embodiment is assumed to include, as a fluorescent probe, phosphor having a fluorescence intensity that varies due to magnetic resonance. In this case, the phosphor can be selected as appropriate on the basis of, for example, the type of the observation target S. For example, in a case where the observation target S is protein in a living body, nanodiamond particles each having an NV center, which are less toxic to living bodies and cause less fading and blinking of fluorescence, are suitably used as phosphor.

The ODMR measurement using the microscope 100A in accordance with the present embodiment will be described.

First, excitation light from the light source 120 is reflected by the dichroic mirror 130 and is radiated through the microscope objective lens 1A including the antenna 10, to the observation target S on the stage 110. Subsequently, light including, for example, fluorescence emitted from phosphor excited by the excitation light and reflected excitation light reflected on the surface of the observation target S travels straight through the microscope objective lens 1A and the dichroic mirror 130, and is then introduced into the bandpass filter 140. Here, a predetermined wavelength band including a fluorescence wavelength from the phosphor is separated by the bandpass filter 140, enters the detector 150 made from, for example, a CCD, and is measured as an amount of fluorescence emission.

Meanwhile, an electromagnetic wave (high frequency such as a microwave) oscillated by the oscillator 200 is transmitted to the antenna 10 provided in the microscope objective lens 1A, and the electromagnetic wave is applied to the observation target S via the antenna 10. In this case, magnetic resonance (electron spin magnetic resonance or nuclear spin magnetic resonance) occurs in the phosphor contained in the observation target S, thereby changing the amount of fluorescence emission. The change in the amount of the fluorescence emission at this time is detected by the detector 150, and a result of the detection is inputted to the process section 160, followed by carrying out required calculation, so that it is possible to acquire information on a change in an amount of fluorescence emission (fluorescence intensity distribution) attributed only to the phosphor and acquire information for grasping dynamics in a living body of the observation target S labeled with the phosphor, from a position in which the phosphor is localized in the living body, movement of the phosphor, and a change in the amount of the fluorescence emission of the phosphor.

It is necessary to apply an electromagnetic wave to the observation target S containing the phosphor in order to carry out the ODMR measurement described above, and a change in an amount of fluorescence emission due to the application of an electromagnetic wave affects the measurement result (measurement accuracy). Therefore, it is important to accurately apply an electromagnetic wave to an observed part of the observation target S. Further, it is important to adjust a position of the antenna 10 under the microscope 100A in order to accurately radiate an electromagnetic wave to the observation target S.

The antenna 10 used in the microscope 100A in accordance with the present embodiment is integrated with the microscope objective lens 1A as described above, and a position of the antenna 10 is adjusted independently of the observation target S on the stage 110.

This enables a relative position of the lens surface 2a and the antenna 10 in the microscope objective lens 1A to be constant and enables positioning of the antenna 10 with the accuracy with which the microscope objective lens 1A is positioned. This enables simple, quick, accurate, highly reproducible positioning of the antenna 10 in applying an electromagnetic wave under a microscope.

Further, the observation target S is placed on the stage 110 and the antenna 10 is integrated with the microscope objective lens 1A, so that it is possible to position the observation target S and the antenna 10 independently of each other. More specifically, moving the stage 110 with the observation target S placed thereon enables a relative position of the observation target S and the antenna 10 to be freely changed, thereby making it easy to dispose the antenna 10 under a part of the observation target S in which the observation is required, so that it is possible to accurately apply an electromagnetic wave to this part.

The oscillator 200 in the microscope 100A in accordance with the present embodiment is configured to wave needed for transmit an electromagnetic observation/measurement, to the antenna 10 included in the microscope objective lens 1A.

As the oscillator 200, a known device that can output an electromagnetic wave can be used. Alternatively, the oscillator 200 may be a combination of a device configured to output an electromagnetic wave and an amplifier.

There is no particular limitation on means for causing the oscillator 200 and the antenna 10 to be connected to or brought into contact with each other to transmit an electromagnetic wave.

Examples of such means include providing a cable 210 directly connecting an end part of the antenna 10 and the oscillator 200, as illustrated in FIG. 3. In this case, an end part of the antenna 10 and the cable 210 may be fixed to each other by, for example, soldering, or alternatively, a clamping tool such as a clip may be provided at a tip of the cable 210 so as to be detachably connected to the antenna 10.

The direct connection between the oscillator 200 and the antenna 10 via the cable 210 makes it possible to efficiently transmit an electromagnetic wave to the antenna 10.

In a typical optical microscope, a plurality of objective lenses are disposed on a revolver, depending on magnifications required for observation. Therefore, in observation/measurement involving change of magnifications, in a case where the antenna 10 and the oscillator 200 are connected to each other directly by the cable 210 for each of a plurality of microscope objective lenses 1A having different magnifications, the increased number of the cables 210 may inhibit rotation of the revolver. Thus, in a microscope 100A including a plurality of microscope objective lenses 1A, it is preferable that an electromagnetic wave is transmitted to the antenna 10 by means other than directly connecting the antenna 10 and the oscillator 200 via the cable 210.

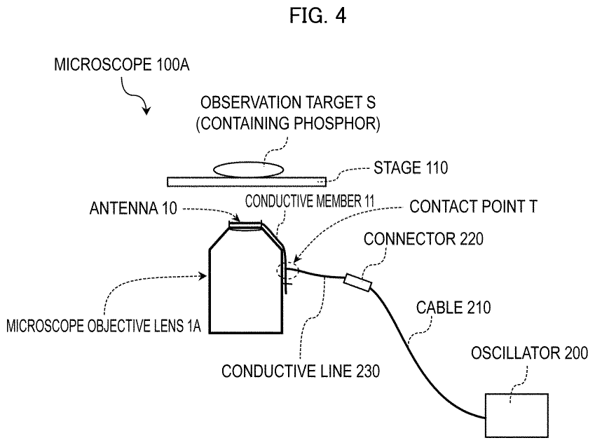

FIG. 4 is an explanatory view schematically illustrating one example of means for transmitting an electromagnetic wave between the oscillator 200 and the antenna 10, as another embodiment of the microscope 100A in accordance with the present embodiment. Note that FIG. 4 is an enlarged explanatory view schematically illustrating a part related to the antenna 10 and the oscillator 200 in the microscope 100A and omits some parts.

The microscope 100A in accordance with the present embodiment illustrated in FIG. 4 includes, as the means for transmitting an electromagnetic wave between the oscillator 200 and the antenna 10, a connector 220 and a conductive line 230 at a tip of the cable 210 connected to the oscillator 200.

In the microscope 100A, when the microscope objective lens 1A is located at an observation position in the microscope 100A (located in a vertically downward direction of the observation target S), the conductive line 230 is disposed at a position that allows the conductive line 230 to form a contact point T with the antenna 10 (conductive member 11) of the microscope objective lens 1A. This makes it possible to transmit an electromagnetic wave to the antenna 10 without inhibiting the rotation of the revolver. In addition, in a case where a magnification of the microscope objective lens 1A is changed without changing an observed part of the observation target S, it is possible to complete the positioning of the antenna 10 in the XYZ directions merely by the rotation operation of the revolver.

As the microscope objective lens 1A used in this case, one including the antenna 10 made from the conductive member 11 extending to a side surface of the housing 3 is preferably used, as illustrated in FIG. 4. This enables the conductive line 230 to be disposed in the vicinity of the side surface of the housing 3 instead of the vicinity of the lens surface 2a, thereby making it easy to secure a space in which the conductive line 230 is disposed.

The conductive line 230 only needs to be one that can transmit an electromagnetic wave from the oscillator 200 through contact with the antenna 10. Examples of the conductive line 230 include a metal line having a larger diameter than that of the conductive member 11 (metal line) used in the antenna 10. This enables the conductive line 230 to have a constant strength and allows for reliable contact with the antenna 10.

Further, the conductive line 230 may be disposed at a fixed position in the microscope 100A or alternatively, may be provided with a moving mechanism that enables movement closer to/away from a side surface of the microscope objective lens 1A. This, when the revolver rotates, causes the antenna 10 and the conductive line 230 to be separated from each other, so that it is possible to prevent a situation in which the conductive member 11 of the antenna 10 is caught by the conductive line 230 and a failure such as disconnection occurs. At the time of microscope observation, it is possible to reliably transmit an electromagnetic wave to the antenna 10 by causing the conductive line 230 to move closer to an antenna 10 side. In the installation of the conductive line 230, it is possible that the conductive line 230 is fixed directly in the microscope 100A or a moving mechanism is provided directly to the conductive line 230, and it is also possible that the connector 220 is fixed or a moving mechanism is provided to the connector 220.

As described above, the microscope 100A in accordance with the present embodiment enables observation/measurement using a microscope objective lens that allows for simple, quick, accurate, highly reproducible positioning of the an electromagnetic wave applying antenna in carrying out observation/measurement involving application of an electromagnetic wave under a microscope. Further, this enables an observation target and the electromagnetic wave applying antenna to be positioned independently of each other, thereby making it possible to apply an electromagnetic wave to a desired part of the observation target.

### Embodiment 2

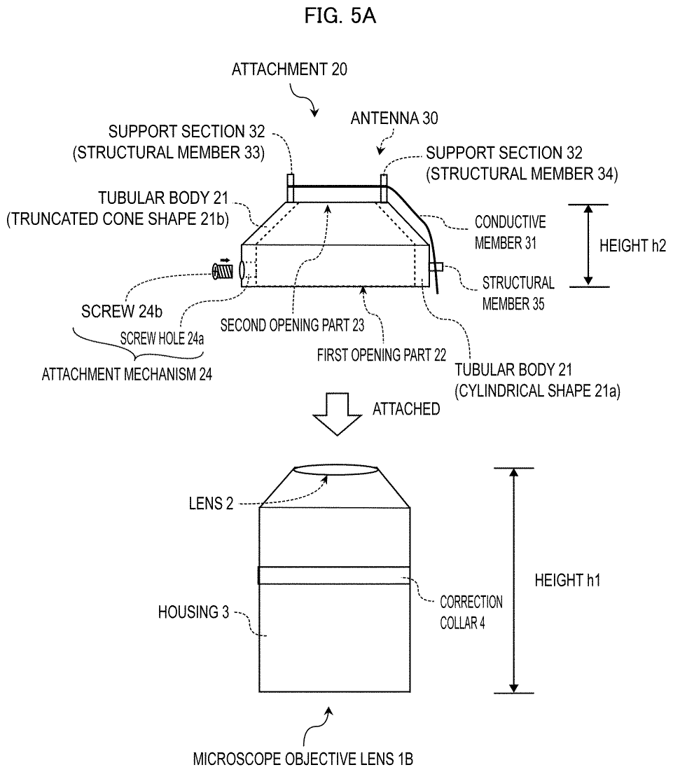

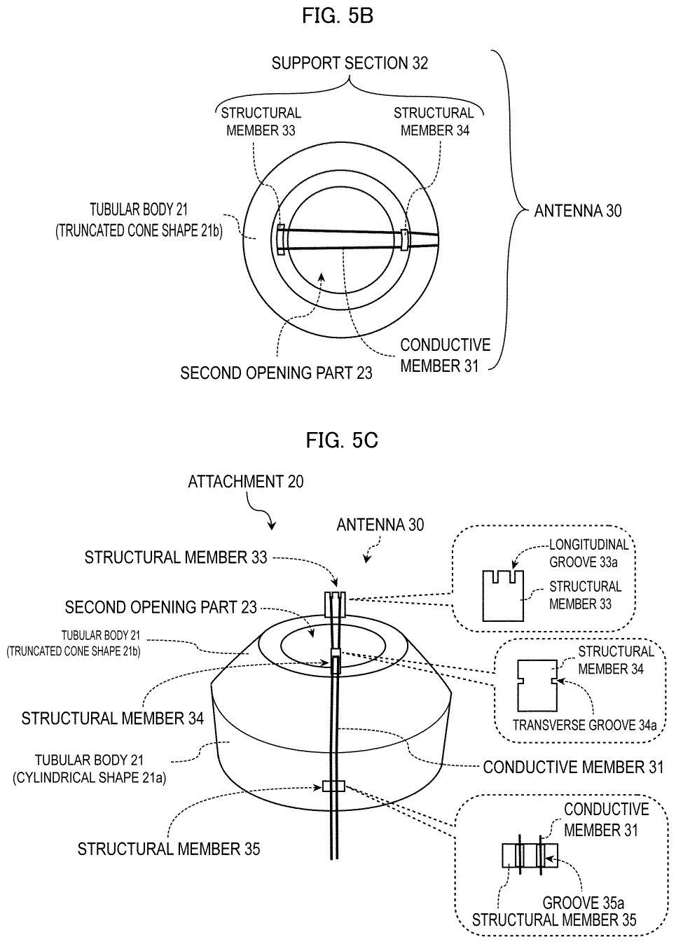

FIG. 5A to FIG. 5C are each an explanatory view schematically illustrating structures of a microscope objective lens and a microscope objective lens attachment each in accordance with Embodiment 2 of the present invention. FIG. 5A is a side view, FIG. 5B is a plan view when seen from above, and FIG. 5C is a perspective view.

The microscope objective lens 1B and the microscope objective attachment 20 in accordance with Embodiment 2 of the present invention are other aspects to fulfil similar functions and effects to those of the microscope objective lens 1A described above. More specifically, an antenna 30 is provided on an attachment 20 side, and the attachment 20 is attached to the microscope objective lens 1B, so that the similar functions and effects to those of the microscope objective lens 1A described above are exerted.

The microscope objective lens 1B in accordance with the present embodiment only needs to include the lens 2 and the housing 3 as illustrated in FIG. 5A, and a known configuration as a microscope objective lens can be used. Further, the microscope objective lens 1B may include a correction collar 4, as illustrated in FIG. 5A.

### Microscope Objective Lens Attachment

The microscope objective lens attachment 20 (hereinafter, also referred to simply as “attachment 20”) in accordance with the present embodiment is an attachment attached to the microscope objective lens 1B.

As illustrated in FIG. 5A to FIG. 5C, the attachment 20 includes a tubular body 21, a first opening part 22 that enables the microscope objective lens 1B to be inserted therethrough into the tubular body 21, and a second opening part 23 that exposes, to an outside, a lens surface 2a of the microscope objective lens 1B inserted into the tubular body 21, the attachment 20 being provided with an antenna 30 formed on a second opening part 23 side.

Attaching the attachment 20 to the microscope objective lens 1B on a lens 2 (lens surface 2a) side of the microscope objective lens 1B from a first opening part 22 side allows the antenna 30 to be disposed on a front side of the lens 2 (lens surface 2a).

The tubular body 21 is a structure including the first opening part 22 and the second opening part 23 and having a shape attachable to the microscope objective lens 1B, and preferably includes an attachment mechanism 24 configured to fix the tubular body 21 to the microscope objective lens 1B.

The tubular body 21 may have any shape that can be attached to the microscope objective lens 1B, and there is no particular limitation on the shape. For example, one possible shape of the cylindrical body 21 is one in which the first opening part 22 having an opening larger than an outer diameter of the microscope objective lens 1B and a second opening part 23 having an opening with an area enough to expose the lens surface 2a to the outside are provided to a tube having both ends with the same diameter and which is fixed to the microscope objective lens 1B by the attachment mechanism 24. In this case, a structure as the entire attachment 20 is simple, thereby facilitating the manufacture thereof.

One example of a shape of the tubular body 21 is, for example, as illustrated in FIG. 5A to FIG. 5C, a shape that includes a cylindrical shape 21a on a first opening part 22 side with an opening larger than an outer diameter of the microscope objective lens 1B and a truncated cone shape 21b on a second opening part 23 side with an opening having an area enough to expose the lens surface 2a to the outside, the truncated cone shape 21b being formed into a shape fitted with a shape of the housing 3 of the microscope objective lens 1B, thereby causing the truncated cone shape 21b to function as the attachment mechanism 24. Another example of the attachment mechanism 24 is, as illustrated in FIG. 5A, one in which a screw hole 24a provided on a side surface of the tubular body 21 (cylindrical shape 21a) and a screw 24b carry out the fixation to the microscope objective lens 1B.

As illustrated in FIG. 5A, the truncated cone shape 21b fitted with the microscope objective lens 1B is included as the attachment mechanism 24 in the attachment 20, and the screw hole 24a and the screw 24b are provided on a side surface of the tubular body 21, so that it is possible to more stably fix the attachment 20 to the microscope objective lens 1B. However, this should not be construed as a limitation. At least one attachment mechanism 24 is preferably provided, and a plurality of attachment mechanisms 24 may be provided.

One possible antenna 30 in accordance with the present embodiment which is formed on a second opening part 23 side is one including a conductive member 31 and a support section 32 supporting the conductive member 31.

The conductive member 31 and the support section 32 supporting the conductive member 31 are provided as the antenna 30, so that it is possible to fix a position and height of the conductive member 31 on the attachment 20, thereby enabling a distance between the antenna 30 and the lens surface 2a of the microscope objective lens 1B and, a distance between the antenna 30 and the focal plane of the observation target S to be stably kept constant. This makes it possible to effectively apply an electromagnetic wave to the observation target S.

The conductive member 31 and the support section 32 can employ similar configurations and structures to those of the conductive member 11 and the fixing section 12 in accordance with the first embodiment, respectively. For example, as illustrated in FIG. 5C, longitudinal grooves 33a and transverse grooves 34a are provided respectively in a structure 33 and a structure 34 as the support section 32, to facilitate support and fixation of the conductive member 31.

The conductive member 31 is used as the antenna 30 in the attachment 20, so that it is possible to easily adjust an intensity of an electromagnetic wave to be applied and directivity of an antenna with a simple process, such as change of a size (area, length, thickness, etc.) of the conductive member 31 and change of an antenna shape formed by the conductive member 31. In particular, since the antenna 30 is formed on the attachment 20, it is possible to safely change the antenna 30 without any risk of touching the lens 2 when the conductive member 31 is changed.

In a case where the conductive member 31 is used as the antenna 30, the conductive member 31 preferably extends to a side surface of the tubular body 21 (cylindrical shape 21a). Extension of the conductive member 31 enables the conductive member 31 and the oscillator 200 to be connected to (brought into contact with) each other at a part on a side surface side of the tubular body 21 (side surface side of the housing 3 of the microscope objective lens 1B). This makes it easy to, even in a case where a working distance as the microscope objective lens 1B is short, secure a space that allows the conductive member 31 and the oscillator 200 to be connected to (brought into contact with) each other.

In this case, as illustrated in FIG. 5A and FIG. 5C, it is preferable to provide a structural member 35 having grooves 35a, to a side surface of the tubular body 21 to fix the conductive member 31 extending to the side surface of the tubular body 21. This enables the conductive member 31 and the oscillator 200 to be stably connected to (brought into contact with) each other.

In light of the fact that the attachment 20 illustrated in FIG. 5A to FIG. 5C is to be attached to the microscope objective lens 1B having the correction collar 4, shown is an attachment 20 in which a height h2 of the cylindrical shape 21a in the tubular body 21 (a height from the first opening part 22 to the second opening part 23) is designed so that when the attachment 20 is attached to the microscope objective lens 1B, a position of the first opening part 22 is located above a position of the correction collar 4 (is designed so as to be not more than one third of a height h1 of the microscope objective lens 1B).

In contrast, it is preferable to make the height of the cylindrical shape 21a higher (longer) in order to facilitate attachment and fixation of the attachment 20 to the microscope objective lens 1B and to, when the conductive member 31 as the antenna 30 extends to a side surface of the tubular body 21 (cylindrical shape 21a) of the attachment 20, stably fix the conductive member 31.

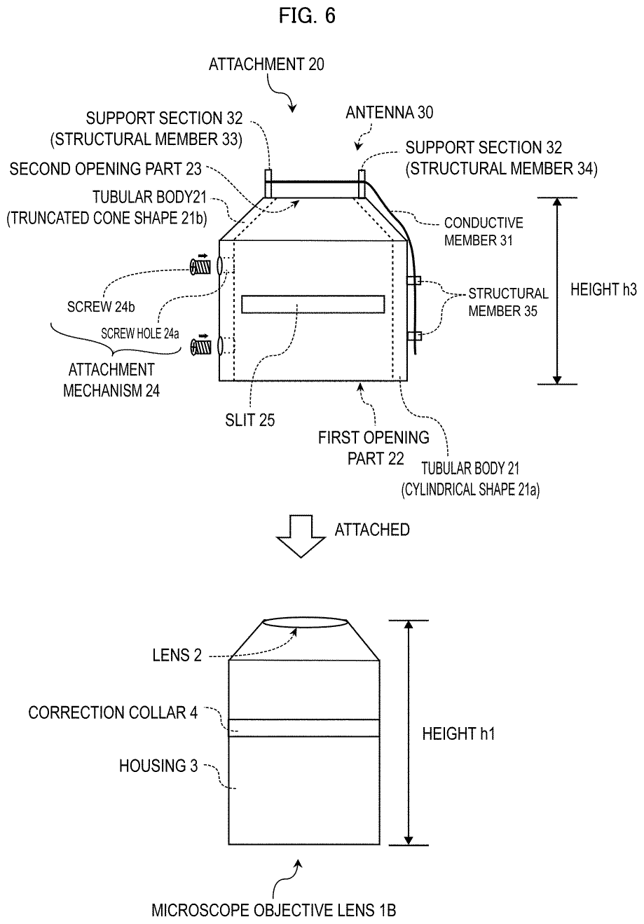

FIG. 6 is an explanatory view schematically illustrating another aspect of the attachment 20 in accordance with the present embodiment.

As illustrated in FIG. 6, a height h3 of the tubular body 21 in the attachment 20 (a height from the first opening part 22 to the second opening part 23) is designed so that when the attachment 20 is attached to the microscope objective lens 1B, a position of the first opening part 22 is located below a position of the correction collar 4 of the microscope objective lens 1B (the height h3 is designed so as to be not less than one third of the height h1 of the microscope objective lens 1B), and in this case, a slit 25 is provided in a circumferential direction of the tubular body 21 (cylindrical shape 21a). This slit 25 is provided at a position corresponding to a position of the correction collar 4 assumed when the attachment 20 is attached to the microscope objective lens 1B.

This makes it possible to utilize advantages achieved by increasing a height of the tubular body 21 and is also advantageous in that operation of the correction collar 4 provided on the housing 3 of the microscope objective lens 1B is not inhibited.

It is preferable to increase the number of the structural members 35 and the number of the attachment mechanisms 24 in accordance with the increase in the height h3 of the tubular body 21, as illustrated in FIG. 6. This makes it possible to more stably fix the attachment 20 to the microscope objective lens 1B and more stably fix the conductive member 31 as the antenna 30.

The antenna 30 is not limited to the one using the conductive member 31, and may be one using a pattern drawn through metal evaporation on a light transmissive substrate. This makes it possible to easily form an antenna having a desired shape. Further, a failure such as disconnection hardly occurs, thereby making it possible to configure an antenna having high durability against repeated use.

The material of the attachment 20 is not particularly limited. Structures other than the conductive member 31 may be made of metal or material other than metal. The attachment 20 is repeatedly used by being attached to the microscope objective lens 1B and therefore is preferably made of material that is light in weight and has a certain degree of a strength. Further, when the attachment 20 expands due to heat, a positional relationship between the antenna 30 and the microscope objective lens 1B is changed. Therefore, the material of the attachment 20 is preferably has a heat resistance (a heatproof temperature of not less than) 100°. Examples of such material include lightweight metals and resins having a heat resistance.

As described above, the microscope objective lens attachment 20 in accordance with the present embodiment enables a relative position of the microscope objective lens 1B with the attachment 20 attached thereto and the antenna 30 to be constant and enables positioning of the antenna 30 with the accuracy with which the microscope objective lens 1B is positioned. This enables simple, quick, accurate, highly reproducible positioning of the electromagnetic wave applying antenna (antenna 30) in applying an electromagnetic wave under a microscope. Further, this enables the observation target S and the electromagnetic wave applying antenna (antenna 30) to be positioned independently of each other, thereby making it possible to apply an electromagnetic wave to a desired part of the observation target S.

Further, the attachment 20 in accordance with the present embodiment can be designed to match a shape of the microscope objective lens, and thus is attachable to various microscope objective lenses. Further, attaching the attachment 20 in accordance with the present embodiment to an existing microscope objective lens (a general-purpose microscope objective lens) makes it possible to easily switch the microscope objective lens between a mode suitable for microscope observation involving application of an electromagnetic wave and a mode suitable for ordinary microscope observation.

The microscope objective lens 1B with the microscope objective lens attachment 20 in accordance with the present embodiment attached thereto is suitably used for microscope observation/measurement involving application of an electromagnetic wave. In particular, it is suitably used as a microscope objective lens for the ODMR measurement.

### Microscope

The microscope objective lens 1B with the microscope objective lens attachment 20 described above attached thereto can be applied to a microscope that carries out observation/measurement by applying an electromagnetic wave to the observation target S.

The following will show an example in which the attachment 20 and the microscope objective lens 1B are applied to an optical microscope (fluorescence microscope) that carries out ODMR measurement, as a microscope of the present invention to which the attachment 20 and the microscope objective lens 1B are applied.

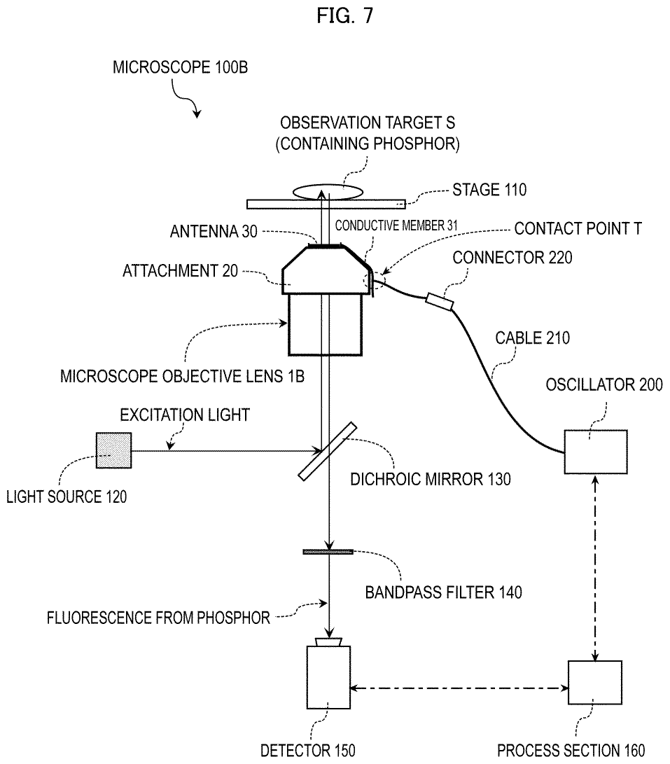

FIG. 7 is an explanatory view schematically illustrating a structure of a microscope in accordance with Embodiment 2 of the present invention. In FIG. 7, the arrow shown by a solid line indicates an optical axis, and the arrow shown by a dot-and-dash line indicates connection that enables input and output.

As illustrated in FIG. 7, a microscope 100B in accordance with the present embodiment includes the microscope objective lens 1B, the attachment 20 including the antenna 30 described above, and the oscillator 200 configured to transmit an electromagnetic wave to the antenna 30. As the attachment 20, FIG. 7 shows one that uses the conductive member 31 as the antenna 30, but this should not be construed as a limitation. It is possible to use ones equivalent to structures described as the microscope objective lens attachment 20 above.

Further, the microscope 100B in accordance with the present embodiment includes structures similar to those of the microscope 100A described above, as a typical structure as an optical microscope (fluorescence microscope). Here, the structures similar to those of the microscope 100A are not described.

In the microscope 100B in accordance with the present embodiment, the microscope objective lens 1A in the microscope 100A described above is replaced with the microscope objective lens 1B with the attachment 20 attached thereto.

Therefore, the observation by the microscope 100B is similar to that by the microscope 100A described above, and thus the detailed description thereof is omitted here.

The oscillator 200 in the microscope 100B in accordance with the present embodiment is configured to transmit an electromagnetic wave needed for observation/measurement, to the antenna 30 included in the attachment 20.

As the oscillator 200, a known device that can output an electromagnetic wave can be used. Alternatively, the oscillator 200 may be a combination of a device configured to output an electromagnetic wave and an amplifier.

There is no particular limitation on means for causing the oscillator 200 and the antenna 30 to be connected to or brought into contact with each other to transmit an electromagnetic wave.

For example, as with the microscope 100A described above, the cable 210 directly connecting an end part of the antenna 30 and the oscillator 200 may be provided, or alternatively, as illustrated in FIG. 7, one including the connector 220 and the conductive line 230 at a tip of the cable 210 connected to the oscillator 200 may be used as means for transmitting an electromagnetic wave between the oscillator 200 and the antenna 30.

Use of the conductive line 230 as the means for transmitting an electromagnetic wave between the oscillator 200 and the antenna 30 as illustrated in FIG. 7 enables transmission of an electromagnetic wave to the antenna 30 without inhibiting revolver rotation, in a case where a plurality of microscope objective lenses 1B with the attachments 20 attached thereto are disposed on a revolver. In addition, in a case where a magnification of the microscope objective lens 1B is changed without changing an observed part of the observation target S, it is possible to complete positioning of the antenna 30 in the XYZ directions merely by the rotation operation of the revolver.

For the material and installation of the conductive line 230, a configuration and structure similar to those in the description of the microscope 100A described above can be employed.

As described above, the microscope 100B in accordance with the present embodiment enables simple, quick, accurate, highly reproducible positioning of an electromagnetic wave applying antenna (antenna 30) in carrying out observation/measurement involving application of an electromagnetic wave under a microscope. Further, this enables the observation target S and the electromagnetic wave applying antenna (antenna 30) to be positioned independently of each other, thereby making it possible to apply an electromagnetic wave to a desired part of the observation target S.

Further, the microscope 100B in accordance with the present embodiment can be used as a microscope that can be easily switched between a mode suitable for microscope observation involving application of an electromagnetic wave and a mode suitable: ordinary microscope observation by attaching and detaching the attachment 20 attachable to the microscope objective lens 1B.

Note that the embodiments described above discuss one example of the microscope objective lens, one example of the microscope objective lens attachment, and one example of the microscope. The microscope objective lens, microscope objective lens attachment, and microscope in accordance with the present invention are not limited to the embodiments described above. The microscope objective lens, microscope objective lens attachment, and microscope in accordance with the embodiments described above may be modified, provided that the gist described in the claims is unchanged.

For example, in the one using a conductive line as an antenna of each of the microscope objective lens and the microscope objective lens attachment in accordance with the present embodiment, the conductive line bridged between the two structural members is shown. However, this should not be construed as a limitation. It is also possible to form the antenna with the increased number of the structural members and the increased number of the conductive lines bridged.

The attachment mechanism in the microscope objective lens attachment in accordance with the present embodiment is not limited to the fixation using the fitting structure or the screw. Another possible example of the attachment mechanism is a mechanism in which an inner diameter of the attachment is changed after the attachment has been attached to the microscope objective lens. For example, one possible configuration is such that a perpendicular slit (opening part) is provided to a part of the tubular body, and a fastener is provided which can close the slit after the attachment has been attached to the microscope objective lens. This enables adjustment with the fastener even for microscope objective lenses having housings with different sizes (outer diameters), thereby making it possible to enhance the versatility in application to existing microscope objective lenses.

### EXAMPLES

The following description will discuss Examples of ODMR measurement carried out with use of a microscope of the present invention. However, the present invention is not limited to only these Examples.

### Microscope

Measurement was carried out with use of a microscope having a structure including the attachment 20 illustrated in FIG. 7. An objective lens having a magnification of 20 times and NA of 0.75 was used as the microscope objective lens 1A. As the dichroic mirror 130, a long-pass dichroic mirror having a cut-on wavelength of 550 nm was used, and as the bandpass filter 140, a long-pass filter having a cut-on wavelength of 650 nm was used. As the light source, a 532-nm solid-state DPSS laser was used, and as the detector 150, a 1,048,576-pixel EMCCD camera was used. In this case, one side of the field of view of the obtained image reached approximately 800 μm. An analog signal generator and a microwave amplifier with a gain of 45 dB which has an amplification band of 1.8 GHz to 4.0 GHz were used as the oscillator 200. An antenna having a structure illustrated in FIG. 1A was used as the antenna 10. An enameled wire having a diameter of 50 μm was used as the conductive member 11.

### Observation Target

HeLa cells were cultivated in a glass bottom dish having a diameter of 35 mm and then were cultivated overnight in a DMEM medium containing 100 μg/ml of nanodiamond, so that the cells were caused to incorporate the nanodiamond, and then were subjected to ODMR measurement after washed with the medium. In this case, Micron+MDA0-0.25 μm, manufactured by Element Six Ltd., which had been subjected to concentration of the NV center by electron beam radiation, was used as the nanodiamond. Further, the cell nuclei were stained with use of Hoechst in order to visually recognize positions of the cells from the fluorescence image.

### ODMR Measurement

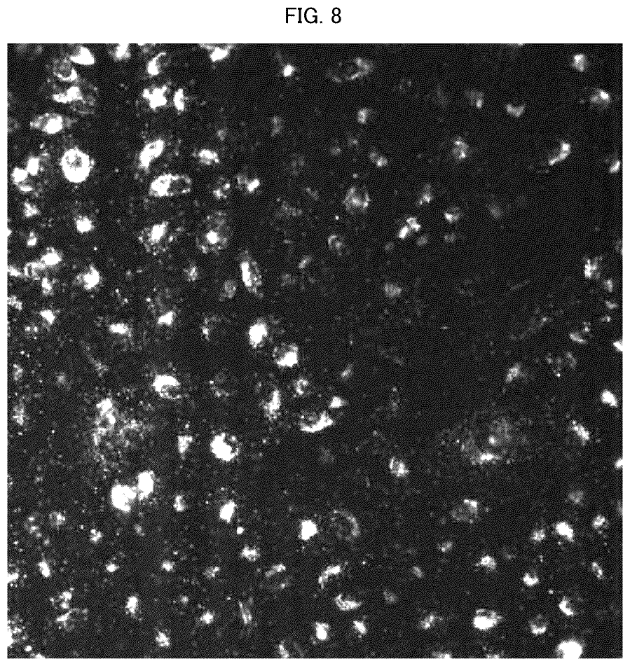

The ODMR measurement was carried out by movie shooting using an EMCCD camera with an exposure time of 42 milliseconds and an EM gain of 10 times. FIG. 8 is a typical image obtained through this movie shooting. The nuclei stained by Hoechst were observed as blue fluorescence, and the nanodiamond incorporated into the cells was observed as red fluorescence. (The rounded shapes in FIG. 8 are the cells, and the white parts in FIG. 8 correspond to the red fluorescence indicating the nanodiamond.) In the scanning in the movie shooting, a fluorescence signal was acquired while the microwave frequency sweep from 2.86 GHz to 2.88 GHz was carried out, in the even-number frames. In this case, the microwave power of the analog signal generator was 9 dBm. In the odd-number frames, a fluorescence signal with no microwave applied was acquired as a reference.

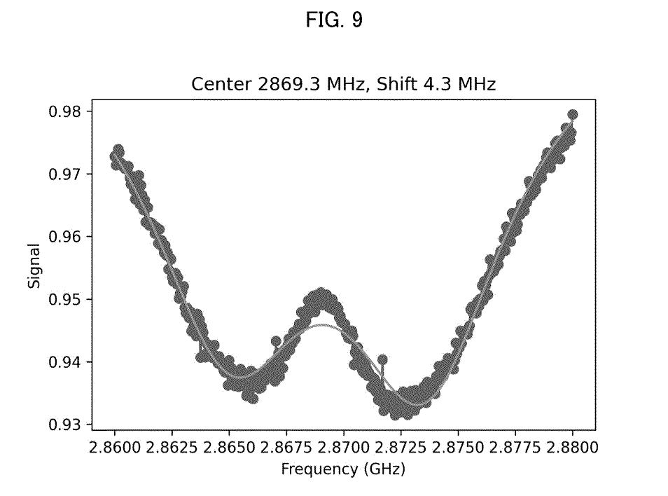

By this measurement, the signal intensity was calculated as a ratio of the fluorescence intensity in the even-number frame with respect to the fluorescence intensity in the odd-number frame. FIG. 9 plots the signal intensity obtained from fluorescence given by a single cell with respect to the swept frequency. This measurement provided a typical ODMR spectrum of the NV center with a bottom around 2.87 GHz.

As described above, use of the antenna 10 having the structure illustrated in FIG. 1A enabled acquisition of the ODMR spectrum over a wide field of view of approximately 800 μm.

### Industrial Applicability

A microscope objective lens, microscope objective lens attachment, and microscope of the present invention are used for microscope observation/measurement involving operation of applying an electromagnetic wave to an observation target. In particular, the present invention is suitably used in measurement through the optically-detected magnetic resonance process.

### Reference Signs List

1A, 1B Microscope objective lens

2 Lens

2a Lens surface

3 Housing

4 Correction collar

10 Antenna

11 Conductive member

12 Fixing section

13, 14, 15 Structural member

13a Longitudinal groove

14a Transverse groove

15a Groove

16 Substrate

17 Pattern

18 Fixing section

20 Attachment

21 Tubular body

21a Cylindrical shape

21b Truncated cone shape

22 First opening part

23 Second opening part

24 Attachment mechanism

24a Screw hole

24b Screw

25 Slit

30 Antenna

31 Conductive member

32 Support section

33, 34, 35 Structural member

100A, 100B Microscope

110 Stage

120 Light source

130 Dichroic mirror

140 Bandpass filter

150 Detector

160 Process section

200 Oscillator

210 Cable

220 Connector

230 Conductive line

h1 Height of microscope objective lens

h2, h3 Height of tubular body of attachment

S Observation target

T Contact point

1. A microscope objective lens comprising an antenna configured to apply an electromagnetic wave to an observation target, the antenna being provided on a front side of a lens surface of the microscope objective lens.

2. The microscope objective lens according to claim 1, wherein the antenna includes a conductive member and a fixing section that fixes the conductive member on a front side of the lens surface.

3. The microscope objective lens according to claim 1, wherein the antenna includes a pattern drawn through metal evaporation on a light transmissive substrate.

4. A microscope objective lens attachment attached to a microscope objective lens, the microscope objective lens attachment comprising:
a tubular body;
a first opening part that enables the microscope objective lens to be inserted therethrough into the tubular body; and
a second opening part that exposes, to an outside, a lens surface of the microscope objective lens inserted into the tubular body,
the microscope objective lens attachment being provided with an antenna that is formed on a second opening part side and that is configured to apply an electromagnetic wave to an observation target.

5. The microscope objective lens attachment according to claim 4, wherein the antenna includes a conductive member and a support section supporting the conductive member.

6. The microscope objective lens attachment according to claim 5, wherein the conductive member extends to a side surface of the tubular body.

7. The microscope objective lens attachment according to claim 4, wherein the antenna includes a pattern drawn through metal evaporation on a light transmissive substrate.

8. The microscope objective lens attachment according to claim 4, wherein the tubular body includes a slit in a circumferential direction.

9. A microscope comprising:
the microscope objective lens according to claim 1; and
an oscillator configured to transmit an electromagnetic wave to the antenna of the

10. A microscope comprising:
a microscope objective lens;
the microscope objective lens attachment according to claim 4; and
an oscillator configured to transmit an electromagnetic wave to the antenna of the microscope objective lens attachment.

11. The microscope objective lens attachment according to claim 5, wherein the tubular body includes a slit in a circumferential direction.

12. The microscope objective lens attachment according to claim 6, wherein the tubular body includes a slit in a circumferential direction.

13. The microscope objective lens attachment according to claim 7, wherein the tubular body includes a slit in a circumferential direction.

14. A microscope comprising:
the microscope objective lens according to claim 2; and
an oscillator configured to transmit an electromagnetic wave to the antenna of the

15. A microscope comprising:
the microscope objective lens according to claim 3; and
an oscillator configured to transmit an electromagnetic wave to the antenna of the microscope objective lens.

16. A microscope comprising:
a microscope objective lens;
the microscope objective lens attachment according to claim 5; and
an oscillator configured to transmit an electromagnetic wave to the antenna of the microscope objective lens attachment.

17. A microscope comprising:
a microscope objective lens;
the microscope objective lens attachment according to claim 6; and
an oscillator configured to transmit an electromagnetic wave to the antenna of the microscope objective lens attachment.

18. A microscope comprising:
a microscope objective lens;
the microscope objective lens attachment according to claim 7; and
an oscillator configured to transmit an electromagnetic wave to the antenna of the microscope objective lens attachment.

19. A microscope comprising:
a microscope objective lens;
the microscope objective lens attachment according to claim 8; and
an oscillator configured to transmit an electromagnetic wave to the antenna of the microscope objective lens attachment.
